(19) United States
(12) Patent Application Publication
(10) Pub. No.: US 2025/0275714 A1
(43) Pub. Date: Sep. 4, 2025

(54) ENHANCING ACCURACY IN WEARABLE SLEEP TRACKERS

(71) Applicant: UNIVERSITY OF SOUTH FLORIDA, Tampa, FL (US)

(72) Inventors: Trung LE, Tampa, FL (US); Phat K. HUYNH, Tampa, FL (US); Lennon TOMASELLI, Tampa, FL (US)

(21) Appl. No.: 19/068,995

(22) Filed: Mar. 3, 2025

Related U.S. Application Data
(60) Provisional application No. 63/560,028, filed on Mar. 1, 2024.

(51) Int. Cl. A61B 5/00

(52) U.S. Cl. CPC A61B 5/4812; A61B 5/7246; A61B 5/7264; A61B 5/7275

(57) ABSTRACT

A system and method for monitoring sleep apnea in cancer patients undergoing treatment are disclosed. The system includes a device to collect SpO2 signals from a patient over multiple sleep sessions, a gateway to receive and process the SpO2 signals to generate formatted SpO2 data, an apnea monitoring service to determine an apnea measure based on the formatted SpO2 data, and a user service to provide a longitudinal progression of the apnea measure. The method involves collecting SpO2 signals, processing them at a gateway, determining an apnea measure, and providing a longitudinal progression of the apnea measure over multiple sleep sessions. The system and method enable efficient monitoring and analysis of sleep apnea in cancer patients during treatment.

### CROSS-REFERENCE TO RELATED APPLICATIONS

This application claims priority to U.S. Provisional Patent Application Ser. No. 63/560,028 filed Mar. 1, 2024, the content of which is hereby incorporated by reference in its entirety.

### BACKGROUND

Wearable devices for monitoring physiological parameters incorporate various sensors to collect data during sleep. These devices may include pulse oximeters for measuring blood oxygen saturation (SpO2), accelerometers for detecting movement, electrocardiogram (ECG) sensors for monitoring heart activity, and temperature sensors. Additional sensors found in some wearable sleep monitoring devices are electromyogram (EMG) sensors for muscle activity, electrooculogram (EOG) sensors for eye movements, and microphones for detecting snoring or abnormal breathing sounds.

Polysomnography is a comprehensive sleep study that records multiple physiological parameters simultaneously. This technique typically measures brain waves (EEG), eye movements (EOG), muscle activity (EMG), heart rhythm (ECG), breathing rate, blood oxygen levels (SpO2), and body position. Polysomnography is conducted in specialized sleep laboratories under the supervision of trained technicians and provides detailed information about sleep architecture, including the identification of different sleep stages and the detection of sleep disturbances.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Aspects of the described technology may provide a method for validating and adjusting a sleep-tracking device. The method includes obtaining polysomnography (PSG) data for a sleep session of a subject, acquiring sensor data from the sleep-tracking device worn by the subject during the sleep session, processing the sensor data to generate an estimated sleep stage time series, performing a statistical correlation analysis between the PSG time series data and the estimated sleep stage time series, calculating a sleep staging accuracy metric based on the correlation analysis, and providing an output to adjust the sleep-tracking device based on the sleep staging accuracy metric. Further aspects may provide a system comprising a processor and a non-transitory computer-readable medium storing instructions that, when executed by the processor, cause the processor to perform operations for validating and adjusting a sleep stage classification process of a sleep-tracking device. Additional aspects may provide a non-transitory computer-readable medium storing instructions that, when executed by a processor, cause the processor to perform operations comprising acquiring sensor data during a sleep session of a subject, applying a trained model to the acquired sensor data to generate a sleep stage classification for the sleep session, and outputting the sleep stage classification, wherein the trained model is trained via a process involving validation against PSG data.

### BRIEF DESCRIPTION OF FIGURES

Non-limiting and non-exhaustive examples are described with reference to the following figures.

FIG. 1 illustrates a flowchart of a method for validating wearable sleep tracking devices, showing steps from data acquisition to device adjustment.

FIG. 2 depicts a series of hypnogram comparisons showing sleep stage data from multiple devices over time, enabling visual comparison of device performance.

FIG. 3 shows a cross-correlation analysis plot displaying the relationship between polysomnography data and device data, illustrating temporal alignment.

FIG. 4 illustrates a flowchart depicting a process for analyzing wearable sleep tracker data, highlighting multi-scale dynamics sampling and accuracy assessment.

FIG. 5 shows a system diagram for a multi-scale dynamical system with reinforcement learning capabilities, featuring an agent with policy and deep Q-network components.

FIG. 6 depicts the neural network architecture of the agent, illustrating input layers, fully-connected layers, and output Q-values for sampling actions.

FIG. 7 illustrates a table describing parameters discussed with respect to FIGS. 5 and 6.

FIG. 8 illustrates a block diagram of a device design process for validating and optimizing wearable sleep tracking devices, featuring a virtual system model and feedback mechanisms.

FIG. 9 illustrates a flowchart of a deep-learning device adjustment process, showing steps from data access to action implementation.

FIG. 10 shows a flowchart depicting a training process for generating a trained agent used in device adjustment.

FIG. 11 illustrates a network diagram showing a system for device analysis and communication, featuring interconnected computing components.

FIG. 12 illustrates a system diagram showing a distributed computing architecture for device analysis, detailing components of data source, computing device, and server.

The foregoing general description of the illustrative embodiments and the following detailed description thereof are merely exemplary aspects of the teachings of this disclosure and are not restrictive.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

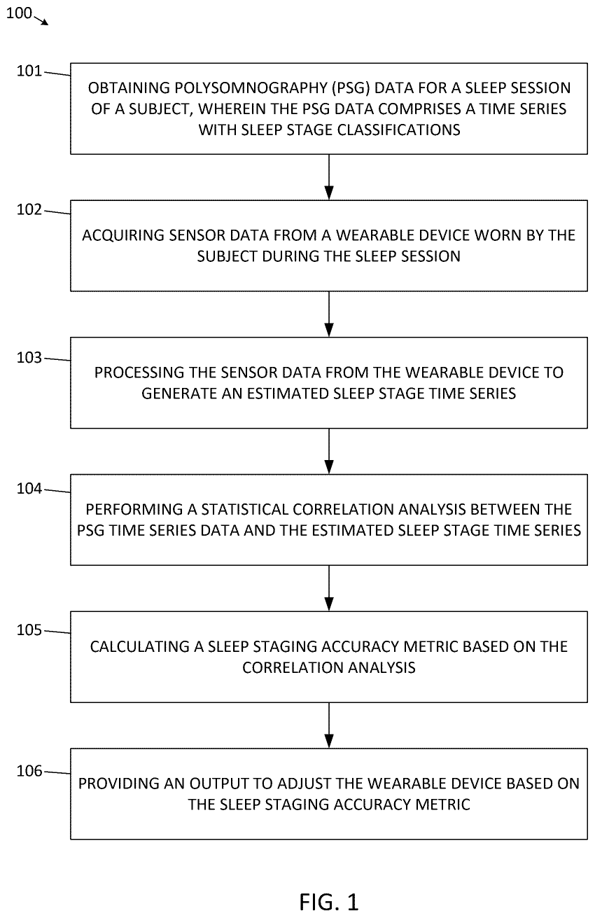

FIG. 1 illustrates a method 100 for validating sleep-tracking devices. Method 100 may comprise a series of steps for analyzing and improving the accuracy of sleep stage classification in sleep-tracking devices. This method may be utilized in various contexts to enhance the performance and reliability of sleep tracking technology. For example, a device manufacturer may employ method 100 during the development and testing phases of new sleep-tracking devices to ensure their products meet industry standards for accuracy. A certification authority might use this method to evaluate and certify different brands of sleep tracking devices, providing consumers with reliable information about product performance. In clinical settings, researchers could apply method 100 to compare the accuracy of consumer-grade sleep-tracking devices against medical-grade polysomnography equipment, helping to bridge the gap between consumer and clinical sleep monitoring. The method may be applied to actual sleep data collected from human subjects wearing both the device under test and polysomnography equipment. Alternatively, it could be used with synthetic data generated by sleep simulation software, allowing for testing of edge cases and rare sleep patterns that might be difficult to capture in real-world studies.

In a step 101, polysomnography (PSG) data may be obtained for a sleep session of a subject. The PSG data may comprise a time series with sleep stage classifications. In some cases, the PSG data may include American Academy of Sleep Medicine (AASM) sleep stage classifications over time. For example, the PSG data may be collected using a clinical-grade polysomnography system that records multiple physiological signals such as brain activity (EEG), eye movements (EOG), muscle activity (EMG), and heart rhythm (ECG) during sleep. The PSG data collection process typically involves attaching various sensors to the subject's body. Electrodes are placed on the scalp to measure brain waves (EEG), near the eyes to detect eye movements (EOG), and on the chin to record muscle activity (EMG). Additional sensors may include nasal airflow sensors, chest and abdominal belts to measure breathing effort, and pulse oximeters to monitor blood oxygen levels.

Once collected, the raw PSG data undergoes several processing steps. First, the data is filtered to remove artifacts and noise. This may involve applying digital filters to remove power line interference or high-frequency muscle artifacts. Next, the cleaned signals are segmented into epochs, typically 30-second intervals, which form the basis for sleep stage classification. Sleep stage classification is then performed on each epoch using standardized criteria, such as those defined by the AASM. This process involves analyzing the characteristics of the EEG, EOG, and EMG signals. For example, the presence of slow, high-amplitude EEG waves (delta waves) is indicative of deep sleep (N3 stage), while rapid eye movements combined with low muscle tone suggest REM sleep.

Automated sleep staging algorithms may be employed to assist in this classification process. These algorithms often use machine learning techniques, such as neural networks or decision trees, trained on large datasets of manually scored PSG recordings. However, the final sleep stage determinations are typically reviewed and validated by trained sleep technicians or clinicians to ensure accuracy. The resulting sleep stage classifications are then compiled into a hypnogram, which provides a visual representation of sleep architecture throughout the night. This hypnogram, along with other derived metrics such as total sleep time, sleep efficiency, and time spent in each sleep stage, forms the basis for clinical sleep assessments and serves as the gold standard for comparison with other sleep tracking methods.

A step 102 may involve acquiring sensor data from a sleep-tracking device worn by the subject during the sleep session. The sleep-tracking device may include various sensors such as accelerometers, heart rate monitors, or temperature sensors. For instance, the sleep-tracking device may be a smartwatch or fitness tracker that collects motion data, heart rate, and skin temperature throughout the night. Various sleep tracking devices may include any sensor without departing from the scope of the described technology. For example, a sleep tracking device might include any or all of an accelerometer, a barometer, a gyroscope, a heart rate sensor, an orientation sensor, an altitude sensor, a cadence sensor, a magnetometer, a blood oxygen sensor, an ambient light sensor, a thermometer, a compass, an impedance sensor, a capacitive sensor, or the like.

A first example device may be a wrist-worn wearable that utilizes a tri-axial accelerometer to measure activity counts at a sampling rate of 32 Hz. This device may produce raw acceleration data in three axes. The first example device may be configured to use different epoch lengths, typically ranging from 15 seconds to 2 minutes, potentially affecting the temporal resolution of the sleep data. Adjusting the sensitivity threshold of the accelerometer may impact how motion is detected and classified, potentially altering the device's ability to distinguish between sleep and wake states.

A second example device may be a wrist-worn smartwatch that combines multiple sensors in a compact form factor. It may include a 3D accelerometer, gyroscope, and body temperature sensor. The accelerometer in the second example device may sample movement at 50 Hz, while the temperature sensor may record data every minute. The gyroscope may provide additional information about hand movements and positioning. The second example device may also incorporate infrared LEDs and photodiodes to measure blood volume pulse at 250 Hz, which may be used to derive heart rate and heart rate variability. Users may adjust the device's sleep detection sensitivity, which may modify the algorithms used to interpret the sensor data for sleep staging.

A third example device may be a more comprehensive sleep tracking smartwatch that may employ a multi-sensor approach. It may feature a 3-axis accelerometer, optical heart rate monitor, SpO2 sensor, and skin temperature sensor. The accelerometer in the third example device may sample at 250 Hz, potentially providing detailed motion data. The optical heart rate sensor may use green, red, and infrared LEDs to measure blood oxygen levels and heart rate variability at a sampling rate of up to 1 kHz during sleep. The SpO2 sensor may sample once per second throughout the night. Users may select different sleep mode settings that may adjust how aggressively the device interprets motion as wake events, potentially impacting the accuracy of sleep stage classification.

A fourth example device may represent a non-wearable option in the form of a thin mat placed under the mattress. This device may utilize pneumatic sensors to detect body movement, breathing rate, and heart rate. The pneumatic sensor may sample pressure changes at 250 Hz, which may then be analyzed to extract respiratory and cardiac signals. The fourth example device may also incorporate a sound sensor that may sample at 4 kHz to detect snoring events. Users may adjust the device's sensitivity to movement, which may affect how it distinguishes between light sleep and wake periods. The placement of the mat under different mattress types may require recalibration to ensure optimal sensor performance.

Lastly, a fifth example device may offer another approach by directly measuring brain activity through a wearable headband. It may use dry EEG electrodes to record brain waves at 250 Hz, potentially providing data similar to that collected in a sleep lab. The fifth example device may also include a pulse oximeter for heart rate and blood oxygen monitoring, as well as an accelerometer for head movement detection. The EEG data may be processed in real-time using onboard algorithms, which may be updated to improve sleep stage classification accuracy. Users may adjust the device's fit and electrode placement, which may significantly impact the quality of the EEG signal and, consequently, the accuracy of sleep stage detection.

In some cases, a comprehensive sleep study may be conducted to simultaneously collect polysomnography (PSG) data and data from multiple wearable sleep tracking devices. The subject may be outfitted with standard PSG equipment, including EEG electrodes, EOG sensors, and EMG electrodes, while also wearing several consumer-grade sleep tracking devices such as smartwatches, fitness bands, and a sleep tracking headband. Additionally, non-wearable sleep tracking devices like under-mattress sensors or bedside monitors may be set up in the sleep laboratory to capture data concurrently with the PSG and wearable devices. Throughout the night, all devices may record data continuously, with the PSG system serving as the gold standard for sleep stage classification while the various consumer devices generate their own sleep metrics and stage estimates. This multi-device approach may allow researchers to directly compare the performance of different sleep tracking technologies against PSG data, potentially revealing strengths and limitations of each device's sensing capabilities and classification algorithms.

In step 103, the sensor data from the sleep-tracking device may be processed to generate an estimated sleep stage time series. This processing may be performed by various components in different configurations. The sleep-tracking device itself may contain an embedded processor and memory storing machine learning models to process the sensor data locally. For example, the device may use a microcontroller running a convolutional neural network to analyze accelerometer and heart rate data and classify sleep stages in real-time. Alternatively, the device may transmit raw sensor data to a user's smartphone or tablet, which can leverage more powerful processors to run more complex sleep stage classification algorithms. The user's computer may execute models stored on its hard drive using systems like TensorFlow or PyTorch to process batches of sensor data and generate sleep stage estimates. In some implementations, the sensor data may be sent to a cloud server for processing. The server may utilize distributed computing resources to run large ensemble models or deep learning networks that analyze data from multiple sensors to produce accurate sleep stage classifications. Some systems may use a hybrid approach, with initial processing done on the wearable device or smartphone, and more intensive analysis performed on a server. The sleep stage classification could also be implemented using specialized hardware. For instance, a neuromorphic chip designed to efficiently run spiking neural networks may be used to classify sleep stages with low power consumption. Application-specific integrated circuits (ASICs) could be developed to rapidly process specific sensor data types and extract relevant sleep-related features. Field-programmable gate arrays (FPGAs) may be configured to implement customized sleep stage classification algorithms optimized for particular device sensors and sampling rates. These hardware implementations may enable faster, more energy-efficient processing compared to general-purpose processors.

Recurrent neural networks (RNNs) or long short-term memory (LSTM) networks may be employed to capture long-term dependencies in sleep patterns. The classification model may be trained on large datasets of labeled polysomnography data to learn the mapping between sensor inputs and sleep stages. Feature extraction techniques like wavelet transforms or spectral analysis may be applied to extract relevant information from raw sensor signals. The model may incorporate multiple sensor inputs, such as accelerometer, heart rate, and skin temperature data, to improve classification accuracy. Ensemble methods combining predictions from multiple models may be used to enhance robustness. The sleep stage estimates may be refined using smoothing techniques to reduce noise and improve temporal consistency. The classification process may adapt to individual sleep patterns over time through online learning approaches. Confidence scores may be generated for each sleep stage prediction to indicate classification reliability. The estimated sleep stages may be aligned with standardized sleep scoring guidelines such as those from the American Academy of Sleep Medicine (AASM). The processing pipeline may include steps for artifact detection and removal to improve data quality prior to classification. Domain expertise may be incorporated into the model architecture or loss function to leverage known sleep physiology. The classification model may be optimized for the specific sensors and sampling rates of the sleep-tracking device under evaluation.

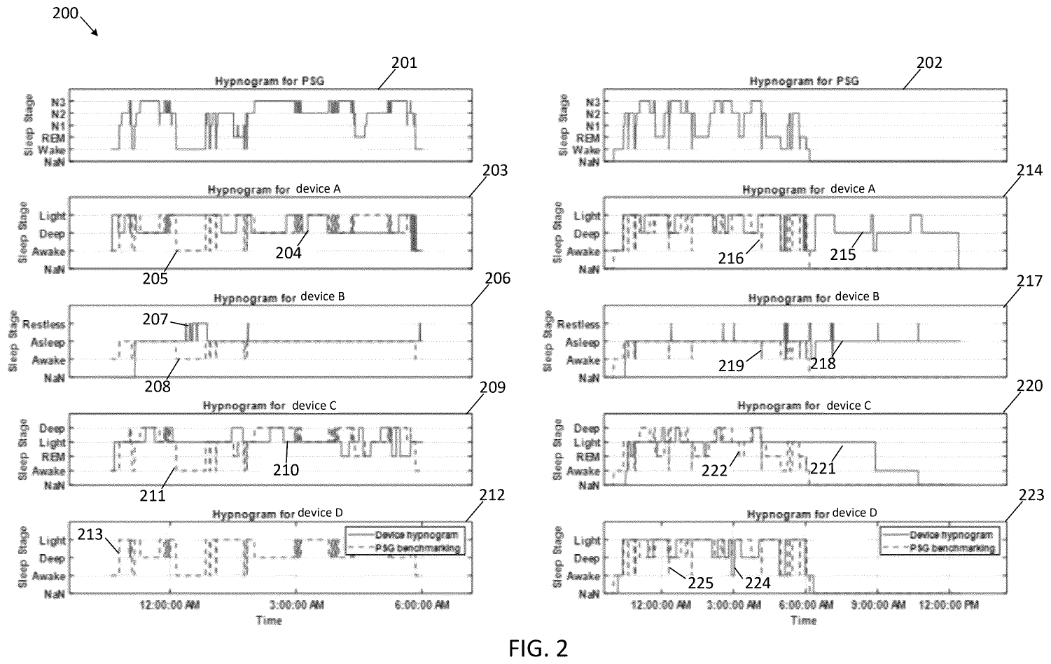

A step 104 may comprise performing a statistical correlation analysis between the PSG time series data and the estimated sleep stage time series. This analysis may involve comparing the sleep stage classifications from the PSG data with the estimated sleep stages from the sleep-tracking device at each time point. The input data for the correlation analysis may include the time-stamped sleep stage labels from both the PSG and device-estimated hypnograms. The analysis may output correlation coefficients, confusion matrices, and graphical representations of the agreement between the two time series. Steps to perform the correlation may include time-aligning the data series, calculating epoch-by-epoch agreement percentages, and applying statistical tests like Cohen's kappa to quantify inter-rater reliability. Advanced techniques such as cross-correlation or wavelet coherence analysis may be employed to identify time-lagged correlations between the PSG and device-estimated sleep stages. FIG. 2 illustrates an example of how this analysis may be performed as part of step 104.

In a step 105, a sleep staging accuracy metric may be calculated based on the correlation analysis. This metric may provide a quantitative measure of how well the sleep-tracking device's sleep stage estimates align with the PSG data. For example, the accuracy metric may include measures such as overall agreement percentage, Cohen's kappa coefficient, or sensitivity and specificity for each sleep stage. The sleep staging accuracy metric calculated in step 105 provides a quantitative assessment of the sleep-tracking device's performance compared to the polysomnography data. This metric serves as an indicator of the device's reliability and validity in sleep stage classification.

The overall agreement percentage offers a straightforward measure of how often the device's classifications match the PSG data across all sleep stages. This metric provides a general sense of the device's accuracy but may not account for agreements occurring by chance. Cohen's kappa coefficient addresses this limitation by measuring the agreement between the device and PSG while accounting for the possibility of chance agreement. A kappa value of 1 indicates perfect agreement, while 0 indicates agreement no better than chance. For sleep stage classification, kappa values above 0.8 are generally considered excellent, 0.6-0.8 good, 0.4-0.6 moderate, and below 0.4 poor. Sensitivity and specificity for each sleep stage provide more detailed insights into the device's performance. Sensitivity measures the device's ability to correctly identify a particular sleep stage when it is present (true positive rate), while specificity measures its ability to correctly identify the absence of a sleep stage when it is not present (true negative rate). These metrics are useful for identifying potential biases in the device's classification algorithm. Additional metrics that may be considered include Pearson correlation coefficient, Spearman's rank correlation coefficient, and cross-correlation function. These accuracy metrics quantify the device's current performance and guide future improvements in sleep stage classification algorithms and sensor configurations.

In addition to correlation analysis, other statistical techniques may be employed to comprehensively evaluate the sleep-tracking device's performance. For instance, resampling methods may be applied to generate hypnogram data at different time scales, allowing for analysis of sleep stage transitions at varying temporal resolutions. The original hypnogram data or underlying raw sensor data may be resampled at different rates, such as aggregating 30-second epochs into 1-minute or 5-minute intervals, or conversely, interpolating to create finer-grained representations. Cross-correlations may then be performed on these resampled hypnograms to assess the agreement between PSG and device-estimated sleep stages across different time scales. Time series analysis techniques, such as autoregressive integrated moving average (ARIMA) models, may be utilized to capture temporal dependencies and predict sleep stage transitions. Multiscale entropy analysis may be applied to quantify the complexity of sleep patterns at different time scales, potentially revealing differences in the device's ability to capture fine-grained versus coarse-grained sleep dynamics. Wavelet coherence analysis may be used to identify time-frequency correlations between PSG and device-estimated sleep stages, highlighting periods of strong or weak agreement across different frequency bands. Additionally, permutation entropy may be calculated to assess the predictability and complexity of sleep stage sequences in both PSG and device-estimated data. These diverse analytical approaches may provide a more nuanced understanding of the sleep-tracking device's performance across various temporal scales and sleep pattern complexities.

A step 106 may involve providing an output to adjust the sleep-tracking device based on the sleep staging accuracy metric. This output may be used to improve the performance of the sleep-tracking device's sleep tracking capabilities. For instance, the output may include recommendations for adjusting sensor sampling rates, modifying feature extraction algorithms, or fine-tuning the sleep stage classification model used by the sleep-tracking device. In some cases, step 106 may comprise outputting the accuracy metric itself or other data indicative of the accuracy of the device. In these cases, step 106 may include adjusting the sleep-tracking device by manually determining adjustments to the device's sensor settings or calibration operations. As another example, step 106 may include adjusting an algorithm for sleep stage classification, updating a machine-learning model based on the accuracy metric or correlation results (e.g., to determine a reward value a deep reinforcement learning process). For example, this data may be uploaded to a computer or transmitted to a remote server for further analysis. The accuracy metric or related data may also be displayed to a user through a mobile application or web interface. These outputs enable continuous refinement and optimization of the sleep tracking device's performance over time.

The output may comprise a comprehensive report detailing the sleep-tracking device's accuracy across various metrics and visualizations. This report may include side-by-side comparisons of the device's hypnogram and the PSG hypnogram, highlighting areas of agreement and discrepancy. Statistical summaries may be presented in tables, showing overall agreement percentages, Cohen's kappa coefficients, and sensitivity/specificity values for each sleep stage. The report may feature time series plots of correlation coefficients, illustrating how the device's accuracy varies throughout the night. Confusion matrices may be included to provide a detailed breakdown of sleep stage classification performance. Heat maps may visualize the wavelet coherence analysis results, showing time-frequency correlations between PSG and device-estimated sleep stages. Multiscale entropy plots may demonstrate the device's ability to capture sleep pattern complexity at different time scales. The report may conclude with actionable insights and recommendations for improving device performance based on the analysis results.

In an example, the steps 101-105 may be applied to different wearable sleep trackers. The resulting report may include a comparative analysis of these devices, presenting their accuracy metrics side by side in a summary table. For instance, the report may include charts, cross-device analyses, statistical test results, device-specific recommendations, ranking of tested devices based on overall performance in sleep stage classification or ability to meet certifications or requirements (e.g., AASM sleep staging requirements), etc.

The output from the sleep staging accuracy analysis may be utilized to generate and refine various aspects of sleep tracking devices. For example, the sleep staging accuracy metric could be used to iteratively adjust device settings through a revision and testing process. The method may be performed repeatedly with different sensor configurations or algorithm parameters. After each iteration, the resulting accuracy metric would be evaluated to determine if performance improved. Based on these results, further adjustments could be made to sampling rates, feature extraction techniques, or classification algorithms. This process may be repeated multiple times, with each cycle potentially yielding incremental improvements to the sleep staging capabilities of the device.

For example, step 106 may include producing outputs such as sleep tracker sensor settings, calibration settings, device configurations, sleep classification models, deep learning models, etc. For instance, sensor settings may be adjusted based on the accuracy metrics, potentially modifying sampling rates or sensitivity thresholds to capture more relevant data. Device settings such as power management or data storage configurations may be optimized to balance accuracy and battery life. Sleep classification models may be fine-tuned or retrained using the insights gained from the correlation analysis, potentially incorporating new features or adjusting model architectures.

Additionally, the output may inform the development of artifact detection algorithms to improve data quality or guide the design of user interfaces to present sleep data more effectively. Another application may involve using the accuracy metrics to calibrate confidence intervals for sleep stage predictions, providing users with a measure of certainty for each classification. These adjustments may be implemented through software updates, firmware modifications, or hardware redesigns, depending on the nature of the improvement. The following three examples illustrate potential adjustments to sleep-tracking devices based on the analysis output:

In one example, the output from the sleep staging accuracy analysis may be used to modify the sensor settings of a wrist-worn sleep tracking device. Based on the calculated Cohen's kappa coefficient indicating moderate agreement (0.55) for REM sleep detection, the device's accelerometer sampling rate may be increased from 32 Hz to 50 Hz during periods of detected low motion, potentially improving the capture of subtle movements associated with REM sleep. Additionally, the heart rate sensor's sampling frequency may be adjusted from once per minute to continuous sampling during the latter half of the sleep period, when REM sleep is more prevalent. The device's gyroscope, previously disabled to conserve battery life, may be activated during these periods to provide supplementary motion data. These sensor modifications may be implemented through a firmware update pushed to the device, with the changes triggered automatically based on the time of night and detected sleep stage transitions. The updated sensor configuration may then be evaluated in subsequent sleep sessions to assess improvements in REM sleep detection accuracy.

In another application, the output may be used to configure the data gathering process and calibrate sensors in a under-mattress sleep tracking system. Analysis of the wavelet coherence results may reveal weak agreement in detecting sleep onset latency, with a 15-minute average discrepancy compared to PSG data. In response, the system's pneumatic pressure sensors may be recalibrated to detect smaller variations in pressure, potentially capturing more subtle indications of sleep onset. The data acquisition system may be configured to increase the sampling rate of the pressure sensors from 100 Hz to 250 Hz during the initial 30 minutes of the recorded session, allowing for more precise detection of the transition from wake to sleep. Additionally, the sound sensor's threshold for detecting movement artifacts may be lowered, enabling the capture of quieter sounds that may indicate wakefulness. These adjustments to the data gathering process and sensor calibrations may be applied through a combination of physical adjustments to the device and software updates to the data processing algorithms. The effectiveness of these changes may be evaluated by comparing the reconfigured system's performance against PSG data in subsequent sleep studies.

The output from the sleep staging accuracy analysis may also be used to update the sleep classification process in a headband-style EEG sleep tracker. Based on the multiscale entropy analysis revealing discrepancies in detecting N3 (deep) sleep transitions, the device's machine learning model for sleep stage classification may be fine-tuned. The existing convolutional neural network architecture may be expanded to include additional LSTM layers, potentially improving the model's ability to capture long-term dependencies in the EEG signal patterns associated with deep sleep transitions. The feature extraction process may be updated to incorporate wavelet transform coefficients, providing more detailed time-frequency information to the classification model. The loss function used during model training may be modified to place greater emphasis on accurately classifying N3 sleep stages, addressing the identified weakness in deep sleep detection. These updates to the sleep classification process may be implemented through an over-the-air software update to the device, with the new model parameters and processing algorithms replacing the previous version. The performance of the updated classification process may then be evaluated using the methods described in steps 101-105, comparing the new results against the previous model's performance and PSG data to quantify improvements in N3 sleep detection accuracy.

The sleep staging accuracy metric may be utilized as a reward signal in a reinforcement learning framework to train and improve sleep stage classification models. In supervised reinforcement learning, the PSG data may serve as the ground truth, guiding the learning process by providing explicit feedback on the model's performance. The agent, which may be implemented as a deep neural network, may learn to map sensor data to sleep stages by maximizing the cumulative reward derived from the accuracy metric. This approach may allow the model to adapt its classification strategy over time, potentially improving its performance as it encounters more diverse sleep patterns. The supervised reinforcement learning method may be particularly effective when high-quality PSG data is available for training, enabling the model to learn from expert-labeled sleep stages.

Analyzing sleep tracking data at multiple time scales may provide a more comprehensive understanding of device performance and sleep patterns. By examining the data at different temporal resolutions, researchers may uncover insights that might be obscured when focusing on a single time scale. For instance, the analysis may involve evaluating sleep stage classifications at 30-second epochs, 5-minute intervals, hourly segments, and whole-night periods. This multi-scale approach may reveal how the device's accuracy varies across different temporal granularities and sleep cycle phases. In some cases, metrics from different time scales could be combined to create more robust performance indicators. For example, a weighted average of Cohen's kappa coefficients calculated at multiple time scales may provide a more balanced assessment of overall classification accuracy. Another approach may involve using the area under the receiver operating characteristic (ROC) curve at various time scales to create a composite metric that reflects the device's performance across different temporal resolutions. Researchers may also consider employing a multi-scale entropy fusion technique, where entropy values calculated at different time scales are integrated to quantify the overall complexity and accuracy of sleep stage detection. Additionally, a time-scale-dependent F1 score may be developed, combining precision and recall metrics from multiple temporal resolutions to provide a comprehensive measure of classification performance.

Method 100 provides a systematic approach for validating and improving sleep-tracking devices by comparing their performance against the gold standard of polysomnography. By iteratively applying this method, manufacturers may continually refine their devices to provide more accurate sleep tracking for users.

FIG. 2 illustrates hypnogram comparisons 200 between polysomnography (PSG) data and various sleep-tracking devices. These comparisons may be used to evaluate the accuracy of sleep tracking devices by visualizing differences in sleep stage classifications over time. FIG. 2 may be illustrative of the type of visualization included in a report in step 106. Additionally, FIG. 2 may be illustrative of data processing operations, such as in steps 103 and 104, where it does not necessarily include an actual visual depiction.

FIG. 2 is described with respect to four example devices. Device A may include an accelerometer sensor to measure body movement. Device B may incorporate an accelerometer sensor and an optical sensor to detect heart rate. Device C may utilize an accelerometer sensor and a bio-impedance sensor to measure multiple physiological parameters. Device D may employ an accelerometer sensor to track motion during sleep. Each device may process the sensor data to generate sleep stage estimates that can be compared to PSG measurements. Of course, the described technology may be applied any number of different devices containing any types of sensors.

The hypnogram comparisons 200 include two PSG hypnograms 201, 202 at the top of each column, which may serve as reference sleep stage classifications. PSG hypnograms 201, 202 may be initially determined with stages including N1, N2, N3, REM, and Wake states plotted on the vertical axis. These PSG hypnograms 201, 202 may represent the gold standard for sleep stage classification, as they are derived from comprehensive polysomnography measurements.

Below the PSG hypnograms 201, 202, comparative hypnograms 203, 214 from a first sleep-tracking device (Device A) are shown. These comparative hypnograms 203, 214 may include device hypnograms 204, 215 and processed PSG hypnograms 205, 216. The device hypnograms 204, 215 may be generated as part of processing the sensor data from the sleep-tracking device, as described in step 103 of method 100. In some cases, generating a device hypnogram 204, 215 may involve applying machine learning algorithms to classify sleep stages based on features extracted from the sleep-tracking device's sensor data.

The next row displays comparative hypnograms 206, 217 from a second sleep-tracking device (Device B), comprising device hypnograms 207, 218 and processed PSG hypnograms 208, 219. Following this are comparative hypnograms 209, 220 from a third sleep-tracking device (Device C), containing device hypnograms 210, 221 and processed PSG hypnograms 211, 222. The bottom row shows comparative hypnograms 212, 223 from a fourth sleep-tracking device (Device D), presenting device hypnograms 224, 225 and processed PSG hypnograms 213.

The processed PSG hypnograms 205, 208, 211, 213, 216, 219, 222, 225 may be derived from the original PSG hypnograms 201, 202 but processed to match the sleep stage classification scheme used by each respective sleep-tracking device. This processing may allow for direct comparison between the PSG data and the sleep-tracking device data.

In some cases, the statistical correlation analysis described in step 104 of method 100 may comprise a cross-correlation analysis between the PSG hypnogram and the sleep-tracking device hypnogram. This analysis may quantify the temporal alignment and similarity between the sleep stages identified by the PSG and those estimated by the sleep-tracking device.

The method for generating and analyzing these hypnogram comparisons 200 may include using statistical programming languages for statistical analysis and autocorrelation function tests. For example, scripts may be developed to perform time series analysis on the sleep stage data, calculating metrics such as agreement percentages, Cohen's kappa coefficients, and lag correlations between the PSG and sleep-tracking device hypnograms. Additionally, numerical computing software may be used for processing raw data and overlaying hypnograms. Signal processing tools may be employed to filter and preprocess the raw sensor data from sleep-tracking devices. Image processing functions may be utilized to create visual overlays of the hypnograms, allowing for easy visual comparison between PSG and sleep-tracking device sleep stage classifications.

The hypnogram comparisons 200 may reveal discrepancies between the sleep stage classifications of different devices and the PSG reference. These discrepancies may be attributed to differences in bio-impedance sensors and varying combinations of sensors used in each device. For instance, some devices may rely primarily on accelerometer data for sleep stage estimation, while others may incorporate heart rate variability or skin temperature measurements.

By analyzing these hypnogram comparisons 200, researchers and device manufacturers may identify specific areas where sleep-tracking devices excel or fall short in accurately classifying sleep stages. This information may be useful for improving the algorithms and sensor configurations used in these devices, ultimately leading to more accurate sleep tracking capabilities in consumer sleep-tracking devices.

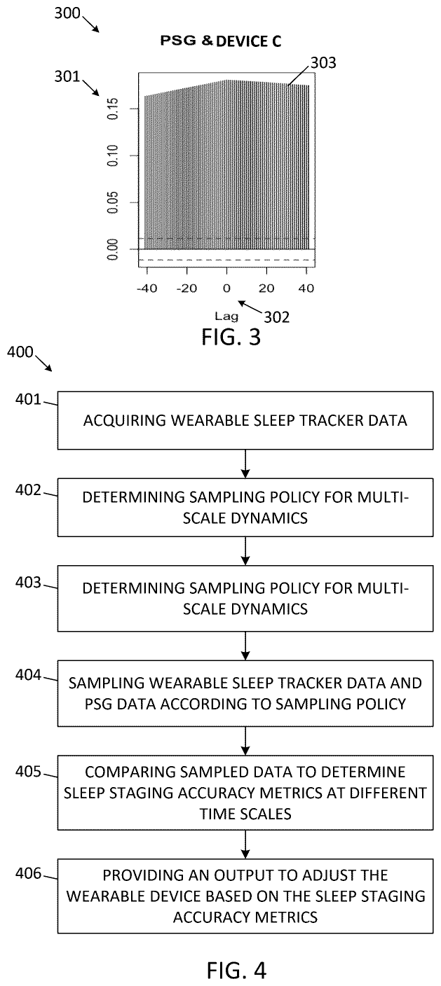

FIG. 3 illustrates a cross-correlation analysis 300 between polysomnography (PSG) data and sleep-tracking device data. Cross-correlation analysis 300 may be used to quantify the temporal relationship between sleep stage classifications from PSG and those estimated by a sleep-tracking device. Cross-correlation analysis 300 includes a correlation axis 301 showing correlation values and a lag axis 302 indicating the temporal offset between the data series. Data points 303 represent the cross-correlation values at different lag times.

The cross-correlation analysis 300 may be based on correlation of hypnogram data of the type illustrated in FIG. 2, or other sleep stage time series datasets. In some cases, the analysis may be performed on sleep stage data from different devices, allowing for comparison between various sleep-tracking technologies. For example, the cross-correlation analysis 300 may be applied to data from Device A, Device B, Device C, and Device D separately, with each device's estimated sleep stages compared against the PSG reference data. This approach may reveal differences in temporal alignment and overall accuracy between devices, potentially highlighting strengths and weaknesses of different sensor configurations or classification algorithms.

The analysis may also be applied multiple times on data sampled at different time scales to provide insights into the devices' performance across various temporal resolutions. For instance, the cross-correlation analysis 300 may be performed on data aggregated into 30-second epochs, 1-minute intervals, or even longer time windows. By comparing the results across these different time scales, researchers may identify whether certain devices perform better at capturing fine-grained sleep stage transitions or broader sleep architecture patterns. This multi-scale approach may offer a more comprehensive understanding of each device's capabilities and limitations in accurately tracking sleep stages over time.

In some cases, cross-correlation analysis 300 may be performed as part of step 104 of method 100, where a statistical correlation analysis is conducted between the PSG time series data and the estimated sleep stage time series. Cross-correlation analysis 300 may provide insights into the alignment and similarity of sleep stage classifications between PSG and sleep-tracking device data over time.

For example, cross-correlation analysis 300 may be implemented using advanced statistical techniques such as time series analysis and signal processing methods. As shown in FIG. 2, the analysis may involve comparing hypnograms from different devices to the PSG reference data. The analysis may involve computing the cross-correlation function between the PSG hypnogram and device hypnogram at various time lags. This process may reveal patterns of agreement or disagreement between the two data sources, helping to identify potential areas for improvement in the sleep-tracking device's sleep stage classification algorithms.

FIG. 4 depicts a process 400 for analyzing sleep-tracking device data using multi-scale dynamics. Process 400 may be an extension of method 100, incorporating analysis at multiple time scales to provide a more comprehensive evaluation of sleep-tracking devices.

Process 400 begins with a step 401 of acquiring sleep-tracking device data. This step may correspond to step 102 of method 100, where sensor data is obtained from a sleep-tracking device worn by a subject during a sleep session. The acquired data may include measurements from various sensors such as accelerometers, heart rate monitors, and temperature sensors.

In a step 402, a sampling policy for multi-scale dynamics may be determined. This step may involve defining a set of time scales at which the sleep-tracking device data and PSG data will be analyzed. For instance, the sampling policy may specify time scales ranging from seconds to hours, allowing for the examination of both fine-grained and coarse-grained sleep patterns.

Step 403 involves sampling sleep-tracking device data and PSG data according to the sampling policy determined in step 402. This step may use advanced statistical and machine learning techniques to analyze actigraphy datasets for algorithm calibration. For example, the sampling process may employ techniques such as wavelet decomposition or multi-resolution analysis to extract relevant features at different time scales.

In step 404, the process compares sampled data to determine sleep staging accuracy metrics at different time scales. This step may involve generating estimated sleep stage time series for each time scale and performing correlation analyses between the sampled PSG data and the estimated sleep stage data. The comparison may utilize machine learning algorithms such as random forests or support vector machines to classify sleep stages based on the sampled data at each time scale.

Step 405 of process 400 involves calculating sleep staging accuracy metrics for each time scale. These metrics may include measures such as overall agreement percentage, Cohen's kappa coefficient, or sensitivity and specificity for each sleep stage at different temporal resolutions. By computing these metrics across multiple time scales, process 400 may provide a more nuanced understanding of the sleep-tracking device's performance in sleep stage classification.

Process 400 concludes with step 406, where an output is provided to adjust the sleep-tracking device based on the sleep staging accuracy metrics from different time scales. This output may include recommendations for modifying sensor sampling rates, adjusting feature extraction algorithms, or fine-tuning sleep stage classification models to optimize performance across various temporal resolutions.

In some cases, process 400 may incorporate cross-sectional studies with diverse sub-populations using biomedical simulators (e.g., a Fluke ProSim simulator or the like) for simulated sleep conditions. These simulators may generate synthetic physiological signals that mimic various sleep disorders or demographic characteristics, allowing for a more comprehensive evaluation of the sleep-tracking device's performance across different populations and sleep conditions.

By performing analysis at multiple time scales and incorporating diverse simulated sleep conditions, process 400 may provide a robust system for validating and improving sleep-tracking devices. This multi-scale approach may enable device manufacturers to optimize their algorithms and sensor configurations for accurate sleep stage classification across a wide range of temporal resolutions and sleep patterns.

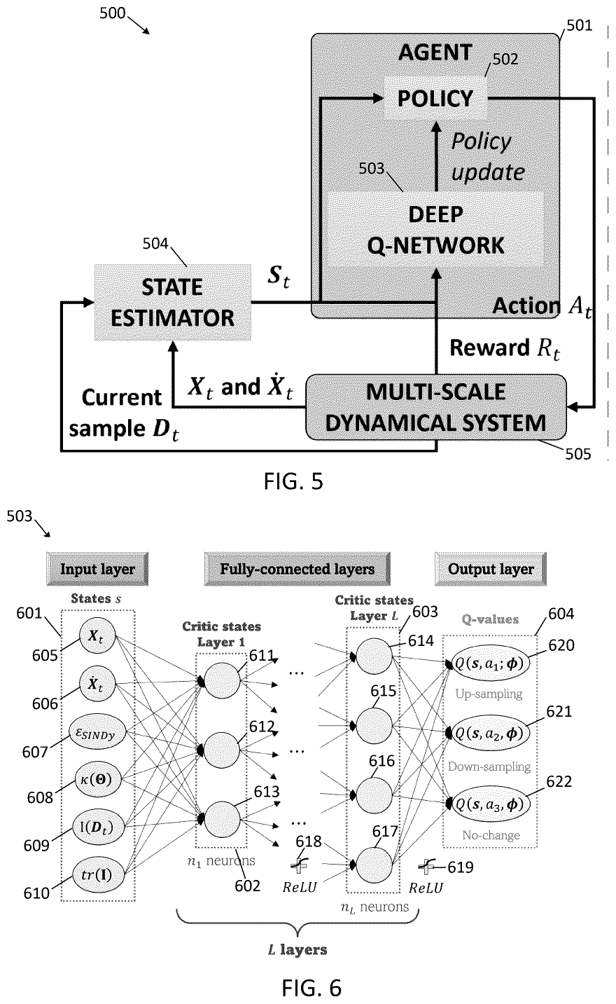

FIG. 5 and FIG. 6 illustrate a multi-scale dynamical system with reinforcement learning capabilities for validating sleep-tracking devices. The system includes a processor executing stored instructions to implement various components. For example, the processor may execute instructions to implement a state estimator 504 that processes current sample data from a data source 505 representing the sleep-tracking device being validated. The state estimator 504 may use techniques such as Kalman filtering or particle filtering to estimate the current state of the sleep tracking system based on incoming sensor data. Additionally, the processor may execute instructions to implement an agent 501 containing a policy 502 and a deep Q-network 503. The agent 501 may be responsible for making decisions about how to adjust the sampling rates and processing of sleep tracking data to optimize accuracy. The deep Q-network 503 may use convolutional neural networks to process time series data from multiple sensors and learn patterns corresponding to accurate sleep stage classifications.

FIG. 5 depicts a model 500 that forms the core of the multi-scale dynamical system. Model 500 may comprise several interconnected components designed to process and analyze sleep tracking data at various temporal resolutions. Agent 501 may contain a policy 502 and a deep Q-network 503, and may be responsible for making decisions about adjusting sampling rates and processing of sleep tracking data to optimize accuracy.

The system may employ an adaptive Runge-Kutta method to solve ordinary differential equations and introduce Gaussian noise to simulate real-world uncertainties. For systems with state measurements from sensors, complete datasets are directly used or state estimation techniques are employed for incomplete data. A low-pass filter reduces high-frequency noise and estimates state derivatives. For highly noise-distorted measurements, total variation regularized derivative estimation methods can provide more accurate estimates. The system develops training experiences for the agent to learn sampling policies, exposing it to various system states and challenges. The policy obtained is then benchmarked against other methods in terms of sample size, robustness to noise, stability of estimated parameters, and sampling time.

Policy 502 within agent 501 may receive updates from deep Q-network 503. Deep Q-network 503 may use deep learning techniques to learn effective actions for different states of the sleep tracking system, employing convolutional neural networks to process time series data from multiple sensors and learn patterns corresponding to accurate sleep stage classifications.

In the context of sleep tracking devices, a controlled Markov process (CMP) may be defined as a tuple ( [symbol] , [symbol] ˜, μ [symbol] (μ⋅, γ), where γ [symbol] ∈[0,1] represents the sleep tracker data (e.g. accelerometer readings, heart rate measurements), [symbol] denotes the sleep stage classification (e.g. wake, light sleep, deep sleep, REM), and π_{ϕ} [symbol] : [symbol] × [symbol] →Δ( [symbol] )a˜π_{ϕ}(⋅|a)ϕ∈Φ⊆ [symbol] ^{m}Π_{Φ} [symbol] ≡M∪ [symbol] is the process for generating a particular sleep stage classification based on a specific sleep tracker data input. The transition model ensures that the next state is drawn as [symbol] : [symbol] s′×˜AP→([−Rₘₐₓ, Rₘₐₓ]⋅|s, a) given the current state s∈ [symbol] (e.g. current accelerometer and heart rate readings) and action a∈ [symbol] , μ: Δ( [symbol] )s (e.g. classifying the current state as light sleep).

$$\text{?} ( Θ ) := \text{?} [ {\sum }_{t = 1}^{∞} {γ}^{t} ℛ ( {s}_{t} , {a}_{t} ) ]$$

$$\text{?} \text{indicates text missing or illegible when filed}$$

Model 500 may include a state estimator 504 that processes current sample data from a data source 505. Data source 505 may represent the sleep-tracking device being validated, providing sensor data at various sampling rates. State estimator 504 may use techniques such as Kalman filtering or particle filtering to estimate the current state of the sleep tracking system based on the incoming sensor data.

The SINDy (Sparse Identification of Nonlinear Dynamics) approach provides a method for analyzing sleep tracker data and enhancing sleep stage classification. This technique enables developers to improve wearable sleep monitoring devices in several ways:
The approach can integrate data from multiple sensors in a sleep tracking device, such as accelerometer, heart rate, and skin temperature. By discovering relationships between various physiological signals and sleep stages, SINDy allows for more comprehensive analysis. The time series nature of sleep data aligns well with the SINDy system, enabling modeling of how different sleep parameters evolve over time and potentially revealing patterns indicative of sleep stage transitions. The library Θ(X) can be constructed using sleep-relevant features like movement intensity, heart rate variability, and respiratory rate, allowing the technique to identify which features are informative for sleep stage classification.

By applying SINDy to data segments around sleep stage transitions, the approach can discover governing equations that describe how physiological parameters change during these transitions. The sparse nature of the discovered equations could allow for personalized sleep models that capture an individual's sleep patterns and physiology. The governing equations discovered by SINDy could be compared with traditional sleep stage classification methods to validate their accuracy and potentially provide new insights into sleep physiology. Additionally, the computational efficiency of SINDy could enable real-time sleep stage classification on resource-constrained wearable devices.

Leveraging these capabilities, sleep tracker developers can potentially improve the accuracy and interpretability of their sleep stage classification methods, leading to more reliable sleep monitoring devices. The SINDy approach offers opportunities for noise reduction, multi-scale analysis, sensor fusion optimization, and adaptive sampling rates. Applying this technology, sleep tracker developers can create devices that provide more accurate sleep stage classification and offer deeper insights into sleep dynamics, personalized sleep optimization, and potential early warning systems for sleep disorders.

The environment in the numerical studies is defined as a two-time-scale deterministic coupled system corrupted by Gaussian noise to represent real-world uncertainties. The system dynamics are characterized by two temporal scales-fast and slow-represented by two sets of variables, respectively denoted asu(t) and v(t). The mathematical form of the deterministic coupled system with the Gaussian noise:

$${τ}_{fasts} {u}^{‵} = f ( u ) + Cv + {ε}_{u} ,$$

$${τ}_{slow} {v}^{‵} = g ( v ) + Du + {ε}_{v} .$$

where εᵤ∈ [symbol] ⁿ and εᵥ∈ [symbol] ^{t} represent the Gaussian noise affecting the states in u(t) and v(t), respectively. They are modeled as multivariate Gaussian distributions:

$${ε}_{u} ∼ 𝒩 ( 0 , diag ( {η}_{u} ) {I}_{n} ) , {ε}_{v} ∼ 𝒩 ( 0 , diag ( {η}_{v} ) {I}_{l} )$$

where ηᵤ∈ [symbol] ⁿ and ηᵥ∈ [symbol] ^{t} represent the variances of the noise associated with the “fast” and “slow” variables. To quantify the impact of noise on the system dynamics, the noise-to-signal ratio (NSR) for each state variable is leveraged. The NSR is calculated as the ratio of the variance of the noise power to the expected signal power. Thus, the NSR for the state variable x∈ [symbol] ᵤ∪ [symbol] ₚ is defined as ηₓ/ [symbol] [x²], where [symbol] [x²] and ηₓ are the expected value and variance of x.

Next, the state space defines the input variables for the Deep Q-network that approximates the state-action-value function Q(s, a). The space [symbol] comprises the SINDy reconstruction error ESINDy, the condition number κ(Θ(Dₜ)) of the matrix Θ(Dₜ), where Dₜ is the current sample of X after the transition t, the multivariate mutual information [symbol] (Dₜ), and the trace of the information matrix tr((Θ(Dₜ)^{T}Θ(Dₜ))⁻¹).

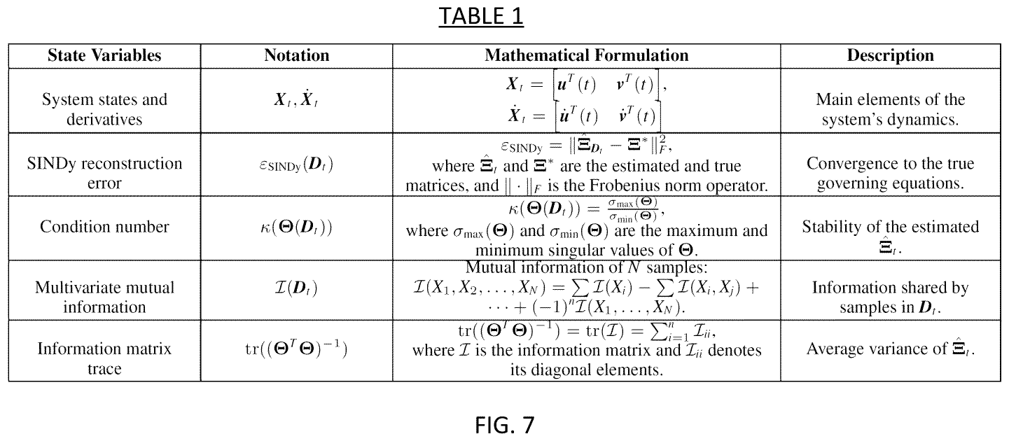

The state space includes input variables that can approximate Q(s, a). Table 1 (FIG. 7) presents these variables, which include state variables, derivatives, and SINDy reconstruction errors for evaluating convergence to governing equations. The condition number assesses matrix stability, while multivariate mutual information analyzes information shared between dataset samples. The information matrix trace calculates average variance using the information matrix and its diagonal elements. These components interconnect through mathematical formulations and data flows to analyze wearable sleep tracking device performance.

The state space for the Q-function, Q(s, a), relates to optimizing wearable sleep tracking device performance through reinforcement learning. This space includes variables for modeling sleep stage dynamics based on sensor data, which may be relevant for applying the reinforcement learning model and evaluating system stability and reliability.

The state space components include direct measurements and calculated derivatives from embedded sensors, such as accelerometric data and heart rate derivatives, which may help capture sleep pattern dynamics for sleep stage classification. Sparse Identification of Nonlinear Dynamical Systems (SINDy) reconstruction errors measure how well the current model fits observed data, serving as a feedback mechanism to refine the model iteratively. Minimizing these errors may enhance predictive model accuracy.

Additional components of the state space include the condition number of estimated matrices, which reflects system output sensitivity to input variations. A lower condition number indicates a more stable system, potentially leading to more consistent performance across different nights and users. Multivariate mutual information assesses shared information between dataset variables, which could be used to reduce problem dimensionality or enhance model predictions by integrating correlated data more effectively. The trace of the information matrix provides a measure of total variance in system parameter estimates, allowing for monitoring of parameter estimate precision and guiding learning process adjustments to improve model accuracy.

The reward signal is designed to encourage the discovery of multi-scale dynamics while maintaining stability and precision in Et estimation. The reward function is defined as:

$${R}_{t} = - {λ}_{κ} log ( κ ( Θ ( {D}_{t} ) ) ) - {λ}_{𝒥} log ( tr ( 𝒥 ) ) - {λ}_{t} t$$

where λ_{κ}, λ [symbol] , and λₜ are non-negative weight coefficients associated with the terms log(κ(Θ(Dₜ))), log (tr( [symbol] )), and the system time t. Here, the condition number κ(Θ(Dₜ)) quantifies a matrix's sensitivity to numerical errors, and its logarithm is included in the reward function to promote stability in the SINDy algorithm. Lower condition numbers contribute to more stable coefficient estimations. The term log (tr( [symbol] )) penalizes high values of the trace of the information matrix, where [symbol] =(Θ(Dₜ)^{T}Θ(Dₜ))⁻¹. Since tr( [symbol] ) represents the inverse of the average variance of the estimated sparse coefficients \grave{Ξ}ₜ, minimizing this term ensures a more reliable parameter estimation. Finally, the inclusion of the time variable t in the reward function incentivizes efficient actions that facilitate faster discovery of multi-scale dynamics, leading to shorter training episodes and higher rewards.

Regarding terminal conditions, a DRL training episode terminates if the number of iterations exceeds a predefined limitEₘₐₓ or if the reconstruction error of the SINDy model, ε_{SINDy}, falls below a specified threshold εₜₒₗ. However, in sleep tracking applications, direct access to the true sparse coefficient matrix Ξ* may be unavailable, making it impractical to compute ε_{SINDy} explicitly. In such cases, an alternative termination criterion is the convergence of the condition number κ(Θ(Dₜ)), which serves as an indicator of numerical stability. A converging κ(Θ(Dₜ)) suggests that the learning process has adequately captured the underlying sleep stage dynamics. This approach can be applied to analyze sleep tracker data by using the condition number as a proxy for model convergence when validating sleep stage classification algorithms.

The creation of the DQN agent involves initializing the critic and target critic that will approximate the value function. The critic is denoted as Q(s, a; ϕ) and the target critic as Qₜ(sₜ, aₜ; ϕₜ), where ϕ and ϕₜ are the critic parameters. The critic and target critic are created as deep neural networks that map the environment states and sampling actions to the expected cumulative reward.

FIG. 6 provides a detailed view of the neural network architecture within agent 501, specifically illustrating a Q neural network 600 used for reinforcement learning. Q neural network 600 may comprise three main sections: an input layer, fully-connected hidden layers, and an output layer.

The input layer of Q neural network 600 may include input states 601 containing various features extracted from the sleep tracking data. These input states 601 may include a sleep data matrix 605, which may contain time series data from multiple sensors in the sleep-tracking device. In some cases, sleep data matrix 605 may include data from bio-impedance sensors in addition to other sensor types such as accelerometers and heart rate monitors.

Input states 601 may also include a derivative matrix 606, which may represent the rate of change of various sleep-related parameters over time. A reconstruction error 607 may be included, quantifying the accuracy of sleep stage reconstructions based on the current model parameters. A condition number 608 may assess the stability and sensitivity of the sleep stage classification algorithms. Mutual information 609 may measure the statistical dependence between different sensor inputs, while an information matrix trace 610 may provide a measure of the overall information content in the input data.

The first layer 602 of Q neural network 600 may contain multiple neurons, including first layer neurons 611, 612, and 613. These neurons may apply weights to the input data and pass the results through activation functions. For example, first layer neuron 611 may use a rectified linear unit (ReLU) activation function to introduce non-linearity into the network. The weights applied by these neurons may be dynamically adjusted during training to optimize the network's performance. Additionally, the first layer may incorporate dropout regularization to prevent overfitting, randomly deactivating a portion of neurons during each training iteration.

The nth layer 603 may represent subsequent hidden layers in the network, containing nth layer neurons 614, 615, 616, and 617. These neurons may use internal activation functions 618 to process data from previous layers. For instance, nth layer neuron 614 may employ a hyperbolic tangent (tan h) activation function to capture complex relationships in the sleep tracking data. The depth of the network, represented by the nth layer, allows for hierarchical feature extraction, with earlier layers capturing low-level features and deeper layers learning more abstract representations. The number of neurons in each layer may be tuned to balance model complexity and computational efficiency. Furthermore, residual connections may be implemented between certain layers to facilitate gradient flow during backpropagation, potentially improving training stability and convergence.

The output layer of Q neural network 600 may produce Q-value outputs 604 corresponding to different actions the agent can take. These may include an up-sample Q 620, representing the value of increasing the sampling rate for one or more sensors, a down-sample Q 621 for decreasing sampling rates, and a no-change Q 622 for maintaining current sampling rates. The output layer may use output activation functions 619, such as softmax, to normalize the Q-values and facilitate action selection.

The multi-scale aspect of the system may be reflected in the ability to process and analyze data at different temporal resolutions. For example, up-sample Q 620 and down-sample Q 621 may allow the system to dynamically adjust sampling rates for different sensors based on the current sleep stage and overall sleep quality. This capability addresses the aspect of modifying sampling rates for at least one of the plurality of sensors as part of the output.

In some cases, the sensor data processed by model 500 may comprise data from a plurality of sensors in the sleep-tracking device, with different sensors having different sampling rates. For instance, an accelerometer may provide data at a higher sampling rate compared to a temperature sensor. The system may learn to optimize these sampling rates independently, potentially increasing the sampling rate of heart rate data during periods of rapid eye movement (REM) sleep while decreasing the sampling rate of motion data during deep sleep stages.

The output of this system may include recommendations for modifying sampling rates, adjusting feature extraction algorithms, and fine-tuning sleep stage classification models based on the learned Q-values and observed performance metrics. These recommendations are derived from the deep reinforcement learning process implemented in the Q neural network, which analyzes the input states and determines optimal actions to improve sleep tracking accuracy.

Modifying sampling rates involves adjusting the frequency at which sensor data is collected from various components of the sleep-tracking device. For example, the system may recommend increasing the accelerometer sampling rate from 32 Hz to 50 Hz during periods of detected movement to capture more detailed motion data, while potentially decreasing the sampling rate of other sensors during periods of inactivity to conserve battery life.

Adjusting feature extraction algorithms refers to refining the methods used to process raw sensor data into meaningful sleep-related features. This may involve implementing new signal processing techniques, such as wavelet transforms or spectral analysis, to extract more relevant information from accelerometer or heart rate data. The system may suggest modifications to these algorithms based on their effectiveness in distinguishing between different sleep stages as determined by the Q-learning process.

Fine-tuning sleep stage classification models involves optimizing the machine learning algorithms responsible for categorizing periods of sleep into specific stages (e.g., light sleep, deep sleep, REM). Based on the learned Q-values, the system may recommend adjustments to model architectures, such as adding LSTM layers to better capture temporal dependencies in sleep patterns, or modifying loss functions to place greater emphasis on accurately classifying specific sleep stages that have shown lower accuracy in comparison to PSG data.

These recommendations are generated by analyzing the performance metrics observed during the validation process, such as the sleep staging accuracy metric and the statistical correlation analysis between PSG data and device-estimated sleep stages. The system continuously refines its recommendations through iterative learning, aiming to optimize the overall performance of the sleep-tracking device across various users and sleep conditions.

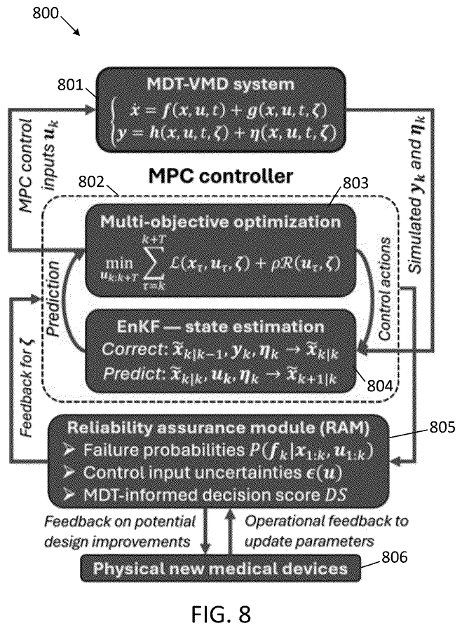

FIG. 8 depicts device design process 800 for validating and optimizing wearable sleep tracking devices. Device design process 800 incorporates interconnected components and processes to enhance accuracy and reliability of sleep tracking devices.

Virtual system model 801 forms the core of device design process 800. Virtual system model 801 represents a Medical Digital Twin-Virtual Medical Device (MDT-VMD) system, described via mathematical equations of state determined based on inputs, including synthetic data and virtual device settings. Virtual system model 801 may include a virtual Auto-adjusting Continuous Positive Airway Pressure (APAP) machine model simulating behavior and interactions of a physical APAP device, allowing comprehensive testing and optimization without physical prototypes.

Device design process 800 includes model predictive control 802 component. Model predictive control 802 receives inputs from virtual system model 801 and performs multi-objective optimization to determine control actions. Model predictive control 802 may dynamically adjust virtual medical device settings in real time based on predictive MDT simulations, optimizing parameters such as pressure levels, flow rates, and response times to maximize sleep quality and minimize discomfort for the simulated patient.

Refinement process 803 incorporates optimization results from model predictive control 802 to adjust system parameters. Refinement process 803 may fine-tune algorithms used for sleep stage classification based on performance of virtual system model 801 under various simulated conditions.

Device design process 800 includes state estimation process 804. State estimation process 804 performs Ensemble Kalman Filter (EnKF)-based state estimation, including correction and prediction of system states. The EnKF approach may be useful for real-time data assimilation from MDTs, allowing continuous updating of virtual system model based on incoming data. State estimation process 804 may use EnKF to estimate current sleep stage of a simulated patient based on multiple noisy sensor inputs, providing a robust estimate compared to single-sensor approaches.

The Reliability Assurance Process 805 may play a role in monitoring and evaluating the overall performance of the sleep tracking system. This process may employ algorithms to calculate failure probabilities for various components, assess uncertainties in control inputs, and generate decision scores informed by the Medical Digital Twin (MDT) simulations. A component of this process may be the Reliability Assurance Module (RAM), which may continuously monitor a range of performance metrics and utilize predictive analytics to anticipate potential system failures before they occur. The RAM may also determine an operational uncertainty value by analyzing the variability and reliability of sensor data over time. This uncertainty value may help quantify the confidence level in the system's measurements and predictions.

The RAM may employ machine learning techniques to analyze patterns in sensor data collected from the sleep tracking device. By examining trends and anomalies in this data, the RAM may identify early warning signs of sensor degradation or impending failure. For example, it may detect subtle changes in accelerometer readings that indicate the sensor may be becoming less sensitive over time, or recognize patterns in heart rate data that suggest the optical sensor may be losing accuracy. The operational uncertainty value may be updated continuously based on these analyses, providing a real-time assessment of the system's reliability.

This predictive capability may allow for proactive maintenance and calibration of the sleep tracking device. When the RAM identifies a potential issue or a rise in operational uncertainty, it may trigger alerts to device manufacturers or users, recommending specific actions such as sensor recalibration, firmware updates, or even device replacement. This approach may help maintain the accuracy and reliability of sleep tracking data over extended periods of use, ensuring that the device continues to provide valuable insights into sleep patterns and potential sleep disorders. The operational uncertainty value may serve as a indicator for determining when such interventions are necessary, helping to optimize the balance between device longevity and data quality.

Furthermore, the RAM's analysis may extend beyond individual sensor performance to evaluate the overall system integrity. It may assess factors such as battery life trends, data transmission reliability, and the consistency of sleep stage classifications over time. By considering these multiple aspects of system performance, the RAM may provide a comprehensive assessment of the sleep tracking device's reliability and effectiveness.

The integration of the RAM within the broader Reliability Assurance Process 805 may enable a dynamic and adaptive approach to sleep tracker validation and optimization. As the system accumulates more data and experiences a wider range of operating conditions, the RAM's predictive models may be continuously refined, leading to increasingly accurate and timely interventions to maintain device performance.

Device design process 800 culminates in sleep tracker adjustment process 806. Sleep tracker adjustment process 806 represents physical new medical devices that can be adjusted based on feedback from reliability assurance process 805. This process may involve updating firmware, recalibrating sensors, or modifying sleep stage classification algorithms based on insights gained from virtual system simulations.

Device design process 800 may involve generating synthetic data stream using virtual system model 801. This synthetic data stream may simulate various sleep patterns, sensor readings, and environmental conditions a physical sleep tracking device might encounter. The system may determine operational uncertainty based on this synthetic data stream, analyzing how well sleep tracking algorithms perform under different simulated conditions, such as varying room temperatures or different sleep disorders.

Device design process 800 may involve determining parameters for a virtual wearable device based on sleep stage classification process and sensor data. These parameters may include sampling rates for different sensors, thresholds for detecting movement or changes in physiological signals, and weights for different features in sleep stage classification algorithm.

Device design process 800 may generate additional synthetic data using a virtual auto-adjusting positive airway pressure (APAP) device. This may allow simulation of complex sleep scenarios, such as those involving sleep apnea treatment. The system may then use virtual wearable device and virtual APAP device to generate a medical condition detection model for a system comprising corresponding physical devices. The model may learn to detect patterns indicative of sleep apnea events based on combined data from simulated wearable sleep tracker and APAP device.

Device design process 800 leverages virtual modeling and synthetic data generation to evaluate and optimize wearable sleep tracking devices. By creating synthetic data stream using virtual system model 801, the process can simulate sleep scenarios a physical device might encounter in real-world use. This approach allows testing device's performance under various conditions without extensive real-world trials.

The synthetic data stream may incorporate multiple variables to create realistic sleep scenarios, simulating different sleep architectures including normal patterns and those associated with sleep disorders. It may include simulated sensor readings mimicking those from accelerometers, heart rate monitors, and other sensors commonly found in wearable sleep trackers. Environmental factors such as ambient light levels, room temperature fluctuations, and background noise can be modeled to test device robustness in different sleep environments.

Analyzing sleep tracking algorithms against synthetic data can determine operational uncertainty. This involves assessing accuracy of sleep stage classification, event detection, and quality metrics under simulated conditions. For example, the system may evaluate how temperature changes affect sleep stage classification accuracy, or how well the device detects micro-awakenings with simulated environmental noise. Determining virtual wearable device parameters based on sleep stage classification and sensor data can help optimize performance. Adjusting parameters like sensor sampling rates allows balancing data resolution and power use. For instance, accelerometer sampling may increase during detected movement and decrease during stillness to conserve battery.

Thresholds for detecting movement or physiological changes are parameters that can be optimized. These affect the device's sensitivity, impacting sleep onset, wake period, and sleep stage transition detection accuracy. Fine-tuning these thresholds based on synthetic data analysis can improve accuracy across sleep patterns and user characteristics. Weights for different features in the sleep stage classification algorithm are another set of parameters. These determine relative importance of inputs like movement, heart rate variability, and skin temperature in classifying sleep stages. Adjusting these weights based on synthetic data performance can optimize classification accuracy. Including a virtual auto-adjusting positive airway pressure (APAP) device in synthetic data generation allows simulating complex sleep scenarios, particularly for sleep apnea treatment. This enables modeling interactions between sleep tracking and therapeutic devices, providing insights into real-world combined operation. Combining virtual wearable sleep tracker and APAP device data can generate a comprehensive medical condition detection model. This model can learn to recognize sleep apnea patterns based on integrated data from both devices. It may correlate tracker-detected movement and heart rate changes with APAP pressure adjustments to improve apnea detection and treatment monitoring.

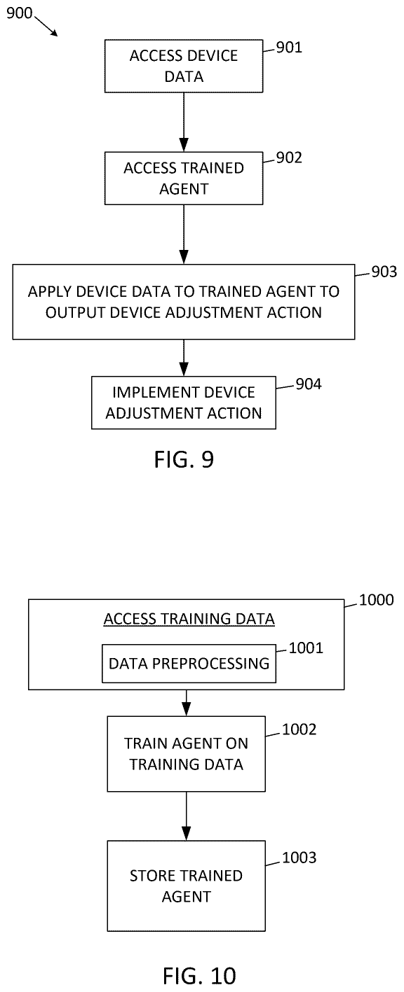

FIG. 9 illustrates a deep-learning device adjustment process 900 for improving the performance of wearable sleep tracking devices. Deep-learning device adjustment process 900 may utilize advanced machine learning techniques to dynamically adjust device parameters based on analyzed data.

Deep-learning device adjustment process 900 may begin with a step 901 of accessing device data. In some cases, step 901 may involve acquiring sensor data during a sleep session of a subject. For example, an accelerometer in a wearable device may collect motion data at 50 Hz, while a photoplethysmography sensor may measure heart rate variability at 1 Hz. This multi-sensor data may provide a comprehensive view of the subject's sleep patterns.

In a step 902, deep-learning device adjustment process 900 may access a trained agent. The trained agent may be a deep neural network that has been previously trained on a large dataset of sleep recordings. For instance, the trained agent may be a convolutional neural network with multiple hidden layers, capable of extracting complex temporal features from the multi-sensor input data.

Deep-learning device adjustment process 900 may proceed to a step 903 where the device data may be applied to the trained agent to output a device adjustment action. In this step, the trained agent may analyze the input data and generate recommendations for adjusting various device parameters. For example, the agent may suggest increasing the sampling rate of the accelerometer during periods of detected movement to capture more detailed motion data.

In a step 904, deep-learning device adjustment process 900 may implement the device adjustment action. This step may involve modifying firmware settings, recalibrating sensors, or updating sleep stage classification algorithms based on the agent's recommendations. For instance, if the agent suggests adjusting the threshold for detecting wake periods, step 904 may involve updating the relevant parameters in the device's sleep scoring algorithm.

FIG. 10 depicts a training process 1000 for generating the trained agent used in deep-learning device adjustment process 900. Training process 1000 may utilize supervised learning techniques to create a model capable of accurately classifying sleep stages and recommending device adjustments.

Training process 1000 may begin with a step 1001 of accessing training data. This step may involve obtaining polysomnography (PSG) data for a training sleep session of a training subject, wherein the PSG data may comprise a time series with sleep stage classifications. Additionally, step 1001 may include acquiring training sensor data from a training wearable device worn by the training subject during the training sleep session. For example, the training data may include EEG recordings from a PSG study along with corresponding accelerometer and heart rate data from a wearable device.

In some cases, step 1001 may also involve data preprocessing operations. These operations may include noise reduction, feature extraction, and data normalization. For instance, a bandpass filter may be applied to the EEG data to isolate frequency bands relevant to sleep stage classification, while accelerometer data may be transformed into activity counts using techniques such as zero-crossing or time-above-threshold methods.

Training process 1000 may proceed to a step 1002 of training an agent on the training data. In this step, a sleep stage classification model may be applied to the training sensor data from the training wearable device to generate an estimated sleep stage time series. The model may use techniques such as recurrent neural networks (RNNs) or long short-term memory (LSTM) networks to capture temporal dependencies in the sleep data.

Step 1002 may also involve performing a statistical correlation analysis between the PSG time series data and the estimated sleep stage time series. This analysis may use metrics such as Cohen's kappa or confusion matrices to quantify the agreement between the model's predictions and the ground truth PSG data. Based on this analysis, a sleep staging accuracy metric may be calculated.

The sleep stage classification model may then be updated based on the sleep staging accuracy metric. This updating process may use backpropagation algorithms to adjust the model's weights and biases, minimizing the discrepancy between the predicted and actual sleep stages. For example, if the model consistently misclassifies REM sleep as light sleep, the weights of neurons responsible for detecting REM-specific features may be adjusted to improve accuracy.

Training process 1000 may conclude with a step 1003 of storing the trained agent. The trained agent, now capable of accurately classifying sleep stages and recommending device adjustments, may be saved in a format suitable for deployment on wearable devices or cloud-based analysis systems.

The combination of deep-learning device adjustment process 900 and training process 1000 may create a powerful system for continuously improving the accuracy of wearable sleep tracking devices. By leveraging large datasets and advanced machine learning techniques, this approach may enable devices to adapt to individual users' sleep patterns and provide increasingly accurate sleep stage classifications over time.

In some cases, the trained model resulting from training process 1000 may be applied to synthetic data streams generated by virtual system model 801 (as described in relation to FIG. 8) to determine operational uncertainty. For example, the trained model may be used to classify sleep stages in simulated data representing various sleep disorders or environmental conditions. The model's performance on these synthetic datasets may provide insights into its robustness and generalizability, helping to identify potential limitations or areas for improvement in the sleep tracking device's algorithms.

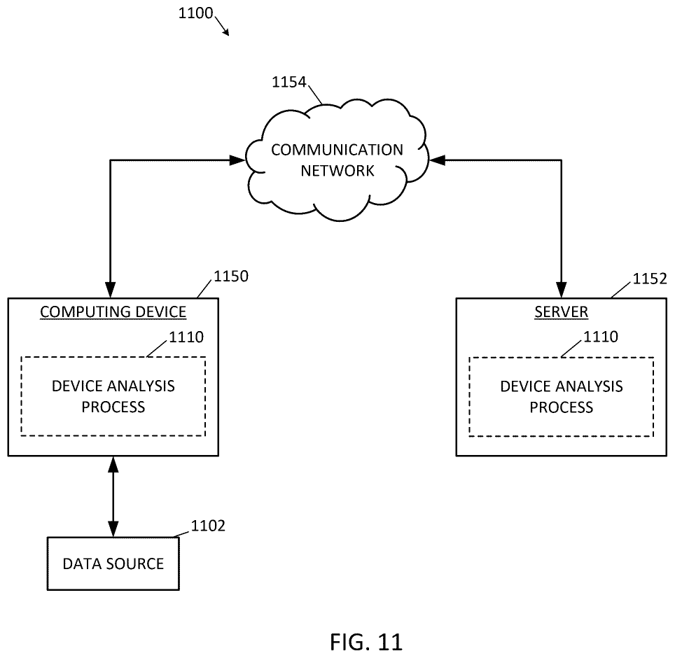

FIG. 11 illustrates a network diagram showing a system 1100 for device analysis and communication. System 1100 may comprise a computing device 1150, a server 1152, and a data source 1102 interconnected through a communication network 1154.

Computing device 1150 and server 1152 may cooperate to perform a device analysis process 1110. In some cases, device analysis process 1110 may involve analyzing data from a wearable sleep tracking device to evaluate and improve its performance. For example, device analysis process 1110 may include steps such as acquiring sensor data, processing the data to generate sleep stage estimates, and comparing these estimates to polysomnography data to calculate accuracy metrics.

Data source 1102 may connect to computing device 1150, allowing data to flow between these components through communication network 1154. In some cases, data source 1102 may represent a wearable sleep tracking device that collects sensor data during a user's sleep session. For instance, data source 1102 may include an accelerometer that measures body movement at a sampling rate of 50 Hz, providing detailed information about sleep-related movements throughout the night.

Similarly, server 1152 may connect to communication network 1154, enabling data exchange with both computing device 1150 and data source 1102. Server 1152 may host more computationally intensive components of device analysis process 1110, such as machine learning models for sleep stage classification or statistical analysis tools for evaluating device performance.

System 1100 may use a star topology, with communication network 1154 serving as a central connection point between computing device 1150, server 1152, and data source 1102. This topology may allow for efficient data transfer and centralized management of the device analysis process. For example, data collected by data source 1102 may be transmitted through communication network 1154 to both computing device 1150 for initial processing and server 1152 for more advanced analysis.

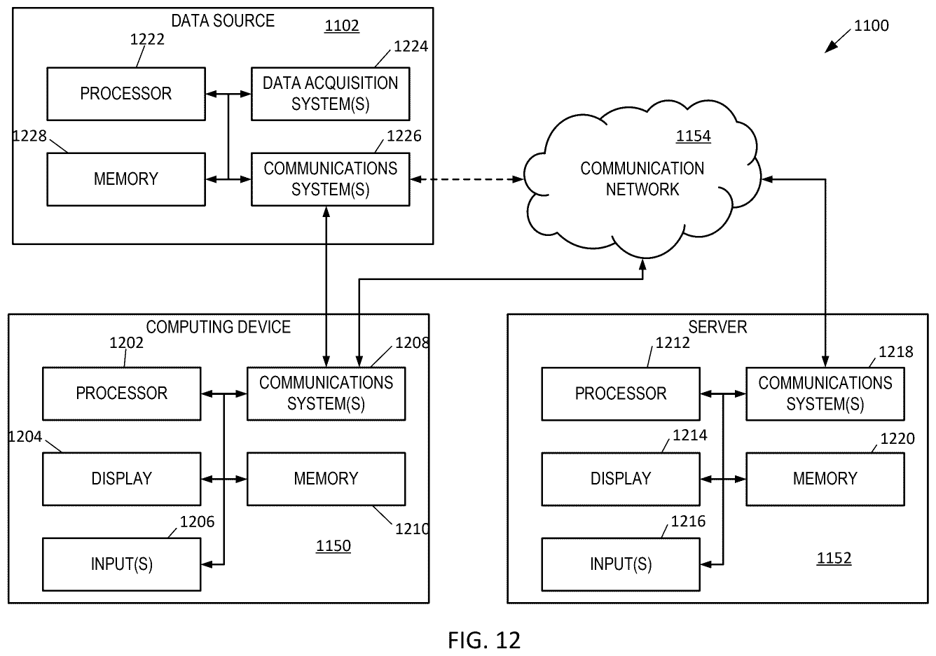

FIG. 12 provides a more detailed view of system 1100, illustrating the internal components of computing device 1150, server 1152, and data source 1102. This figure shows how these components interact to facilitate the device analysis and communication process.

Computing device 1150 may include a computing processor 1202, a display interface 1204, an input interface 1206, a computing communications system 1208, and computing memory 1210. Computing processor 1202 may process data received through computing communications system 1208. For example, computing processor 1202 may execute algorithms to preprocess raw sensor data from a wearable sleep tracking device, such as applying noise reduction techniques or extracting relevant features for sleep stage classification.

Display interface 1204 may provide visual output, such as graphical representations of sleep stage data or performance metrics for the wearable device. Input interface 1206 may accept user inputs, allowing researchers or device manufacturers to interact with the analysis process. For instance, a user may input parameters for adjusting sensor settings or calibration through input interface 1206.

Computing memory 1210 may store data and instructions for computing processor 1202. In some cases, computing memory 1210 may contain software modules that implement various components of device analysis process 1110, such as data preprocessing routines or statistical analysis tools.

Server 1152 may contain a server processor 1212, a server display 1214, a server input 1216, a server communications system 1218, and server storage 1220. Server processor 1212 may execute data analysis operations, such as running complex machine learning models for sleep stage classification. Server display 1214 may provide output visualization, potentially showing more detailed or aggregate results from the device analysis process.

Server input 1216 may accept control inputs, allowing administrators to manage the analysis process or update analysis algorithms. Server communications system 1218 may manage network connectivity, facilitating the exchange of large datasets or analysis results with computing device 1150 and data source 1102. Server storage 1220 may maintain data and processing results, potentially storing historical performance data for multiple wearable devices over time.

Data source 1102 may comprise a source processor 1222, a data acquisition system 1224, a source communications system 1226, and source memory 1228. Source processor 1222 may control the operation of various sensors in the wearable sleep tracking device. Data acquisition system 1224 may interface with source processor 1222 to collect data from these sensors.

For example, data source 1102 may be implemented in a wrist-worn sleep tracking device with the following components: a source processor 1222 such as an ARM Cortex-M4 running at 80 MHz to manage sensor data collection and processing; a data acquisition system 1224 with interfaces for multiple sensors including a 3-axis accelerometer sampling at 50 Hz (e.g. 25-100 Hz) to detect motion, an optical heart rate sensor sampling at 1 Hz (e.g. 0.5-2 Hz), a skin temperature sensor sampling every 5 minutes (e.g. 1-10 minutes), and an ambient light sensor sampling at 1 Hz (e.g. 0.5-2 Hz); a source communications system 1226 with Bluetooth Low Energy (BLE) 5.0 module for transmitting collected data to a paired smartphone; and source memory 1228 with 64 MB flash memory (e.g. 32-128 MB) to store up to 7 days (e.g. 3-14 days) of sensor data and device settings. This implementation of data source 1102 enables comprehensive sleep data collection through multiple sensor types while maintaining a compact, wearable form factor suitable for continuous overnight use.

For example, data acquisition system 1224 may include interfaces for multiple sensor types commonly found in wearable sleep tracking devices. These may include an accelerometer for measuring body movement, a barometer for detecting changes in altitude or pressure, a gyroscope for measuring orientation, and a heart rate sensor for monitoring cardiovascular activity during sleep. In some cases, data acquisition system 1224 may also interface with more specialized sensors such as a blood oxygen sensor for detecting sleep apnea events, or a capacitive sensor for measuring skin conductance as an indicator of sleep quality.

Source communications system 1226 may enable data transmission from data source 1102 to other components of system 1100. For instance, source communications system 1226 may use Bluetooth Low Energy (BLE) protocols to transmit collected sensor data to computing device 1150 at regular intervals or upon request. Source memory 1228 may store collected data temporarily before transmission, as well as configuration settings for the various sensors.

The interconnected components of system 1100 may work together to facilitate comprehensive analysis and improvement of wearable sleep tracking devices. For example, data collected by data source 1102 may be transmitted through communication network 1154 to computing device 1150 for initial processing. Computing device 1150 may then send the preprocessed data to server 1152 for more advanced analysis, such as comparing the device's sleep stage classifications to polysomnography data.

Based on the results of this analysis, system 1100 may generate recommendations for adjusting the wearable device. These adjustments may include modifying sensor settings or calibrations to improve accuracy. For instance, if the analysis reveals that the accelerometer in data source 1102 is not sensitive enough to detect subtle movements during light sleep, system 1100 may recommend increasing the accelerometer's sampling rate or adjusting its sensitivity threshold.

In some cases, the reconstruction error 607, previously discussed in relation to the Q neural network 600, may be analyzed across the distributed system components of system 1100. For example, computing device 1150 may calculate initial reconstruction error values based on the preprocessed sensor data, while server 1152 may perform more detailed analysis of how this error relates to overall sleep staging accuracy. The results of this analysis may then be used to fine-tune the sleep stage classification algorithms or sensor configurations in data source 1102.

By leveraging the distributed architecture and specialized components of system 1100, researchers and device manufacturers may continuously improve the accuracy and reliability of wearable sleep tracking devices. The system's ability to collect, process, and analyze data from various sensor types, combined with its capacity for sophisticated statistical and machine learning analyses, may enable rapid iteration and optimization of these devices for enhanced sleep monitoring capabilities.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “system,” “engine,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on). For instance, as shown in FIG. 2, the edge device 202 includes various components such as sensors 208, protocol interfaces, gateway interface 238, software processes including model 288 and analysis service 274. These components work together to collect and transmit SpO2 data, demonstrating how multiple components can be integrated within a single system to perform complex functions.

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

A number of implementations have been described. Nevertheless, it will be understood that various modifications may be made without departing from the spirit and scope of the disclosure. Accordingly, other implementations are within the scope of the following claims.

1. A method, comprising:
obtaining polysomnography (PSG) data for a sleep session of a subject, wherein the PSG data comprises a time series with sleep stage classifications;
acquiring sensor data from a sleep-tracking device worn by the subject during the sleep session;
processing the sensor data from the sleep-tracking device to generate an estimated sleep stage time series;
performing a statistical correlation analysis between the PSG time series data and the estimated sleep stage time series;
calculating a sleep staging accuracy metric based on the correlation analysis; and
providing an output to adjust the sleep-tracking device based on the sleep staging accuracy metric.

2. The method of claim 1, wherein acquiring sensor data from the sleep-tracking device comprises obtaining data from at least one of: an accelerometer, a barometer, a gyroscope, a heart rate sensor, an orientation sensor, an altitude sensor, a cadence sensor, a magnetometer, a blood oxygen sensor, an ambient light sensor, a thermometer, a compass, an impedance sensor, or a capacitive sensor.

3. The method of claim 1, wherein:
the sensor data comprises data from a plurality of sensors of the sleep-tracking device;
sensor data from a first one of the plurality of sensors corresponds to a first sampling rate and sensor data from a second one of the plurality of sensors corresponds to a second sampling rate different from the first sampling rate; and
the output comprises a modified sampling rate for at least one of the plurality of sensors.

4. The method of claim 1, further comprising:
for each respective time scale of a plurality of time scales: sampling the sensor data and the PSG data at a sampling rate based on the respective time scale to generate respective time series sensor data and respective sampled PSG time series data;
identifying respective features of the respective time series sensor data;
generating a respective estimated sleep stage time series based on the respective features;
performing a respective statistical correlation analysis between the respective sampled PSG time series data and the respective estimated sleep stage time series; and
calculating a respective sleep staging accuracy metric based on the respective correlation analysis; and
providing the output based on the plurality of sleep staging accuracy metrics.

5. The method of claim 1, wherein:
processing the sensor data comprises generating a sleep-tracking device hypnogram;
the PSG time series data comprises a PSG hypnogram; and
the statistical correlation analysis comprises a cross-correlation analysis between the PSG hypnogram and the sleep-tracking device hypnogram.

6. The method of claim 1, further comprising:
determining parameters for a virtual sleep-tracking device based on the sleep stage classification process and the sensor data;
generating a synthetic data stream using the virtual sleep-tracking device; and
determining operational uncertainty based on the synthetic data stream.

7. The method of claim 6, further comprising:
generating additional synthetic data using a virtual auto-adjusting positive airway pressure (APAP) device; and
using the virtual sleep-tracking device and the virtual APAP device to generate a medical condition detection model for a system comprising a physical sleep-tracking device corresponding to the virtual sleep-tracking device and a physical APAP device corresponding to the virtual APAP device.

8. The method of claim 6, wherein determining the operation uncertainty comprises applying a trained deep-learning model to the synthetic data stream.

9. The method of claim 1, wherein the PSG time series data and the estimated sleep stage time series each comprise American Academy of Sleep Medicine (AASM) sleep stage classifications over time.

10. The method of claim 1, wherein adjusting the sleep-tracking device comprises adjusting a sensor setting or adjusting a sensor calibration.

11. A system comprising:
a processor; and
a non-transitory computer-readable medium storing instructions that, when executed by the processor, cause the processor to:
obtain polysomnography (PSG) data for a sleep session of a subject, wherein the PSG data comprises a time series with sleep stage classifications;
acquire sensor data from a sleep-tracking device worn by the subject during the sleep session;
process the sensor data from the sleep-tracking device using a sleep stage classification process to generate an estimated sleep stage time series;
perform a statistical correlation analysis between the PSG time series data and the estimated sleep stage time series;
calculate a sleep staging accuracy metric based on the correlation analysis; and
provide an output to modify the sleep stage classification process of the sleep-tracking device based on the sleep staging accuracy metric.

12. The system of claim 11, wherein acquiring sensor data from the sleep-tracking device comprises obtaining data from at least one of: an accelerometer, a barometer, a gyroscope, a heart rate sensor, an orientation sensor, an altitude sensor, a cadence sensor, a magnetometer, a blood oxygen sensor, an ambient light sensor, a thermometer, a compass, an impedance sensor, or a capacitive sensor.

13. The system of claim 11, wherein:
the sensor data comprises data from a plurality of sensors of the sleep-tracking device;
sensor data from a first one of the plurality of sensors corresponds to a first sampling rate and sensor data from a second one of the plurality of sensors corresponds to a second sampling rate different from the first sampling rate; and
the output comprises a modified sampling rate for at least one of the plurality of sensors.

14. The system of claim 11, wherein the instructions, when executed by the processor, further cause the processor to:
for each respective time scale of a plurality of time scales:
sample the sensor data and the PSG data at a sampling rate based on the respective time scale to generate respective time series sensor data and respective sampled PSG time series data;
identify respective features of the respective time series sensor data;
generate a respective estimated sleep stage time series based on the respective features;
perform a respective statistical correlation analysis between the respective sampled PSG time series data and the respective estimated sleep stage time series; and
calculate a respective sleep staging accuracy metric based on the respective correlation analysis; and
provide the output based on the plurality of sleep staging accuracy metrics.

15. The system of claim 11, wherein:
processing the sensor data comprises generating a sleep-tracking device hypnogram;
the PSG time series data comprises a PSG hypnogram; and
the statistical correlation analysis comprises a cross-correlation analysis between the PSG hypnogram and the sleep-tracking device hypnogram.

16. The system of claim 11, wherein the instructions, when executed by the processor, further cause the processor to:
determine parameters for a virtual sleep-tracking device based on the sleep stage classification process and the sensor data;
generate a synthetic data stream using the virtual sleep-tracking device; and
determine operational uncertainty based on the synthetic data stream.

17. The system of claim 16, wherein the instructions, when executed by the processor, further cause the processor to:
generate additional synthetic data using a virtual auto-adjusting positive airway pressure (APAP) device; and
use the virtual sleep-tracking device and the virtual APAP device to generate a medical condition detection model for a system comprising a physical sleep-tracking device corresponding to the virtual sleep-tracking device and a physical APAP device corresponding to the virtual APAP device.

18. The system of claim 16, wherein determining the operation uncertainty comprises applying a trained deep-learning model to the synthetic data stream.

19. The system of claim 11, wherein the PSG time series data and the estimated sleep stage time series each comprise American Academy of Sleep Medicine (AASM) sleep stage classifications over time.

20. A non-transitory computer-readable medium storing instructions that, when executed by a processor, cause the processor to perform operations comprising:
acquiring sensor data during a sleep session of a subject;
applying a trained model to the acquired sensor data to generate a sleep stage classification for the sleep session; and
outputting the sleep stage classification,
wherein the trained model is trained via a process comprising:
obtaining polysomnography (PSG) data for a training sleep session of a training subject, wherein the PSG data comprises a time series with sleep stage classifications;
acquiring training sensor data from a training sleep-tracking device worn by the training subject during the training sleep session;
applying a sleep stage classification model to the training sensor data from the training sleep-tracking device to generate an estimated sleep stage time series;
performing a statistical correlation analysis between the PSG time series data and the estimated sleep stage time series;
calculating a sleep staging accuracy metric based on the correlation analysis; and
updating the sleep stage classification model based on the sleep staging accuracy metric.
